SEQUENCE LISTING (2 sequences)

SEQ ID NO: 1 - DNA, 21 bp, Artificial Sequence
  forward primer
catccagagcgtccctggctt

SEQ ID NO: 2 - DNA, 20 bp, Artificial Sequence
  reverse primer
ggccgaagaaacactgagaa

(19) United States
(12) Patent Application Publication
(10) Pub. No.: US 2019/0225958 A1
(43) Pub. Date: Jul. 25, 2019

(54) METHOD FOR ENRICHMENT AND PURIFICATION OF CELL-FREE DNA FROM BODY FLUID FOR HIGH-THROUGHPUT PROCESSING

(71) Applicant: JBS Science Inc., Doylestown, PA (US)

(72) Inventors: Jamin D. Steffen, Yardley, PA (US); Surbhi Jain, Doylestown, PA (US); Wei Song, Audubon, PA (US)

(73) Assignee: JBS Science Inc., Doylestown, PA (US)

(21) Appl. No.: 16/253,952

(22) Filed: Jan. 22, 2019

Related U.S. Application Data
(60) Provisional application No. 62/620,678, filed on Jan. 23, 2018.

(51) Int. Cl. C12N 15/10; C12Q 1/686; C12Q 1/6806; C12Q 1/6834

(52) U.S. Cl. CPC C12N 15/1003; C12Q 1/686; C12Q 2600/112; C12Q 1/6834; C12Q 1/6806

(57) ABSTRACT

Disclosed is a method for the enrichment and purification of circulating, cell-free DNA (cfDNA) from a biological specimen. The method uses optimized mixtures and ratios of magnetic particles and reagents to efficiently enrich, purify, and isolate cfDNA that can be useful for cancer detection and monitoring for precision medicine. The method can be used for optimized detection of infectious diseases. The versatility of the method enables both manual use and high-throughput automation use. A kit for the use of this method is also disclosed.

### CROSS REFERENCE TO RELATED APPLICATION

This application claims priority to U.S. Provisional Application No. 62/620,678 filed on Jan. 23, 2018. The content of the provisional application is incorporated herein by reference in its entirety.

### GOVERNMENT INTERESTS

This invention was made with government support under R43HG008700 and R44HG008700 awarded by National Institutes of Health. The government has certain rights in the invention.

### FIELD OF THE INVENTION

The disclosures herein relate to reagents and methods for isolation of circulating cell-free DNA from a body fluid. The present invention relates to methods of isolating, enriching and purifying mutated or altered DNA from a body fluid for clinical diagnosis, precision medicine, liquid biopsy, and monitoring of therapeutic efficacy. This invention can be designed for coupling with high-throughput processing for shortening isolation time and for robotic automation.

### BACKGROUND

Circulating cell-free DNA (cfDNA) has been found in several bodily fluids, and serves as an excellent source of DNA-based biomarkers for the detection and monitoring of cancer [1]. Plasma is commonly used for liquid biopsy to acquire cfDNA, which circumvents the need for invasive tissue biopsy, although other sources of cfDNA (such as urine and saliva) are trending to become an even more accessible way to collect and identify tumor biomarkers. Nevertheless, the highly fragmented nature, low abundance, and contaminating wild-type (or non-tumor) DNA have presented major obstacles in developing biomarkers using circulating cfDNA. It is evident that a robust and versatile method that can enrich and purify cfDNA from one body fluid collection will be important in the future of liquid biopsy [2]. Isolation of nucleic acid from biological specimens using chaotropic agents is common practice. However, optimal cfDNA isolation methods that provide abundant, enriched marker DNA are currently being pursued. There is a need for a more robust and versatile method for enriching or purifying cfDNA.

### SUMMARY

This invention addresses the need mentioned above in a number of aspects.

In one aspect, the invention provides a method of isolating or enriching circulating cell-free DNA (cfDNA) molecules. The method comprises (a) providing a solution containing DNA molecules; (b) contacting the solution with a silica-based substrate under a binding condition allowing low molecular weight DNA (LMW DNA) molecules and high molecular weight DNA (HMW DNA) molecules to bind to the silica-based substrate; (c) eluting the silica-based substrate to obtain an eluate; (d) contacting the eluate with a first carboxylated-based substrate under a first condition allowing the HMW DNA molecules to bind to the first carboxylated-based substrate; and (e) obtaining unbound DNA molecules, thereby isolating or enriching the LMW DNA and/or cfDNA molecules.

In this method, the binding condition may comprise about 5-20 mM (such as 5-20, 6-18, 7-15 mM, and 10 mM) EDTA. The binding condition may further comprise one or more of the following: a chaotropic agent and alcohol. The obtaining step may comprise (i) contacting the unbound DNA molecules with a second carboxylated-based substrate under a second condition allowing the cfDNA molecules, not allowing impurity that affecting PCR reaction, to bind to the second carboxylated-based substrate and (ii) eluting the second carboxylated-based substrate to obtain the purified cfDNA molecules. The second condition may comprise PEG (e.g., PEG-8000), isopropanol, TWEEN 20, and a salt. One or more of the substrates can be in the form of beads, membrane, or a column. Eluting the silica-based substrate or carboxylated-based substrate comprises eluting with water or a low-salt TE buffer.

In another aspect, the invention provides method of isolating or enriching cfDNA molecules. The method comprises (a) providing a solution containing DNA molecules; (b) contacting the solution with a first silica-based substrate under a first condition allowing HMW DNA molecules to bind to the silica-based substrate; (c) obtaining unbound DNA molecules; (d) contacting the unbound DNA molecules with a second silica-based substrate under a second condition allowing cfDNA molecules to bind to the second silica-based substrate; and (e) eluting the second silica-based substrate to obtain an eluate, thereby isolating or enriching the cfDNA molecules.

In this method, the first condition may comprises about 20-40 mM (such as 25-35 and 30 mM) EDTA. The first condition may further comprise one or more of a chaotropic agent and alcohol. The second condition may comprise free of EDTA, an EDTA reversal agent, or pH about 5-7 (such as 5.5, 6.0, and 6.5). Examples of the reversal agent include a divalent cation (e.g., Mg²⁺ or Ca²⁺) and an acidic agent. The second condition may further comprise a chaotropic agent. One or more of the substrates can be in the form of beads or a column. Eluting the substrate comprises eluting with water or a low-salt TE buffer.

In all the methods described above, the solution containing DNA molecules can be prepared from a biological sample of a subject (e.g., a human or a non-human animal). Examples of the sample include blood, urine, or other samples form. In some embodiments, the biological sample has been concentrated to reduce the volume thereof. For example, the concentration can be carried out by centrifugation. To stabilize cfDNA and prevent degradation, the biological sample can be treated with EDTA and TRIS within a few minutes post collection from the subject.

In a further aspect, the invention provides a kit for isolating or enriching cfDNA molecules. The kit comprises (i) a silica-based substrate and (ii) one or more selected from the group consisting of a carboxylated-based substrate, EDTA, a chaotropic agent, an alcohol, an eluting buffer, PEG, a detergent, and an EDTA reversal agent.

The details of one or more embodiments of the invention are set forth in the description below. Other features, objectives, and advantages of the invention will be apparent from the description and from the claims.

### BRIEF DESCRIPTION OF THE DRAWINGS

FIG. 1 is a flow chart that depicts the overall process of a 2-bead method (option A) for enrichment of cfDNA from body fluid. Once the body fluid is collected, it may be treated immediately (or within 5 minutes) with up to 20 mM EDTA and TRIS buffer for stability. Body fluid may undergo concentration by filter centrifugation if a smaller volume is desired. Body fluid (concentrated or not-concentrated) is treated with a chaotropic agent (such as guanidine thiocyanate), an alcohol (such as isopropanol) and silica-based magnetic beads. After 30 minutes, the supernatant is removed; beads are washed with 85% ethanol, and eluted to yield total DNA. This elution is fractionated and further purified with 0.285× carboxylated-based magnetic beads to bind DNA greater than 1 Kb in size. The supernatant is collected and mixed with PEG-8000, TWEEN-20, NaCl, TRIS pH 7.5, and carboxylated-based magnetic beads. The beads are washed with 85% ethanol and eluted in a low salt TE buffer to provide enriched and further purified cfDNA that is less than 1 Kb is size.

FIG. 2 is a flow chart that depicts the overall process of a 1-bead method (option B) for enrichment of cfDNA from body fluid. Once the body fluid is collected, it may be treated immediately (or within 5 minutes) with 20-40 mM EDTA and TRIS buffer for stability. Body fluid may undergo concentration by filter centrifugation if a smaller volume is desired. Body fluid (concentrated or not-concentrated) is then mixed with a chaotropic agent (such as guanidine thiocyanate), an alcohol (such as isopropanol) and silica-based magnetic beads. After 30 minutes, the unbound is collected, as the beads will size selectively bind larger DNA fragments based on how much EDTA is present. The unbound is treated with a chaotropic agent (such as guanidine thiocyanate) and silica-based magnetic beads in the presence of an EDTA reversal agent. An EDTA reversal agent can include divalent cations (such as MgCl₂ or CaCl₂) or acidic reagents that bring the mixture pH to below 6.0. The addition of an EDTA reversal agent allows the cfDNA to bind to the magnetic beads, which can then be washed with 85% ethanol and eluted in low salt TE buffer. The isolated DNA can be further purified using DNA purification solution using carboxylated beads.

FIG. 3 is a photograph showing comparison of a JBS Urine cfDNA isolation kit with other currently available cfDNA isolation kits. Equivalent fractions of a large urine collection were provided for 5 cfDNA isolation kits. These kits include the JBS Urine cfDNA isolation kit, ZYMO Research Quick-DNA Urine Kit, ABNOVO cell-free DNA isolation Kit, intron cell-free DNA isolation kit, and NEXTPREP-Mag cfDNA isolation kit. For all kits the protocol was followed for cfDNA isolation of 40 mL of urine. A cfDNA aliquot (equivalent of 10 mL urine) was added to each lane of an agarose gel and compared. The JBS DNA isolation kit is optimal in that it provides purified cfDNA less than 1 Kb in size. Other kits provide cfDNA of low size, but are also contaminated with larger cellular DNA (above 1 Kb) that is not from circulating DNA and interferes with detection. MW: Molecular Weight; bp: base pairs; Kb: Kilobase pairs; HMW: High-Molecular Weight; LMW: Low-Molecular Weight.

FIG. 4 is a photograph showing concentration of urine volume from 20 to 80-fold reduction. Urine was concentrated at various amounts and DNA was isolated. The isolated DNA were added to a lane on an agarose gel and compared. Concentration up to 80-fold has no effect on DNA recovery or size selection.

FIG. 5 is a photograph showing optimal amounts of chaotropic agent (guanidine thiocyanate) and alcohol (isopropanol) for efficient recovery of DNA from silica-based magnetic bead isolation. Varying equivalents of 2 M Guanidine thiocyanate were added to 0.5 mL of 40× concentrated urine. Also, varying amounts of isopropanol were also added (displayed as final percentage) to compare optimal recovery. At least 2 equivalents of 2 M guanidine thiocyanate at 25-75% isopropanol were found to be the optimal conditions. * indicates approximate nucleosomal size; All depicted nucleosomal sizes are less than 1 Kb as shown.

FIG. 6 is a photograph showing Option B method of described cfDNA enrichment. Equivalent amounts of urine DNA were collected and treated with either 20 or 40 mM of EDTA, as depicted. The cfDNA isolation method was followed according to option B, and unbound portion was treated with 10-100 mM of MgCl₂ and bound to the second round of beads. Varying amounts of EDTA between 20 and 40 mM can influence the size selection of LMW DNA left in the unbound of the first silica-based magnetic bead binding. This inhibition of LMW DNA binding beads can be reversed by divalent cations (such as Magnesium and Calcium) or by decreasing the pH below 6.0.

FIG. 7 is a photograph showing urine treatment with dry powder EDTA in TRIS pH 8.0. Equivalent Urine aliquots were obtained and treated immediately post collection with up to 20 mM EDTA and TRIS pH 8.0, or a powder form that dissolves in liquid to reach up to 20 mM EDTA in TRIS pH 8.0. Dissolution may take anywhere from 2-30 minutes depending on sample type. Treated 10 mL urine was left sit at RT for 5 days, in duplicates (1 and 2). The urine samples were then run through the JBS Isolation protocol (without HMW removal) to provide total isolated urine DNA. An aliquot of this elution was run on an agarose gel.

FIGS. 8A, 8B and 8C are a set of diagrams showing results of detecting (A) a Y chromosome DNA in a plasma sample from a pregnant female with a male fetus; (B) a spiked 107 bp double stranded HBV PCR product; and (C) DNA encoding the 18 S ribosomal RNA before and after cleanup with carboxylated beads.

FIG. 9 is a diagram showing results of detecting a 107 bp double stranded (ds) HBV PCR product in a human plasma sample before and after cleanup with carboxylated beads.

FIG. 10 is a diagram showing results of detecting and quantifying a 107 bp double stranded HBV PCR product in human urine samples by qPCR assay before and after cleanup with carboxylated beads.

### DETAILED DESCRIPTION OF THE INVENTION

This invention relates to reagents and methods for isolation of cfDNA. The usefulness of cfDNA isolation applies to detection of tumor formation or presence, detection of fetal DNA, or detection of infectious organism DNA. Downstream applications of cfDNA include Next Generation Sequencing (NGS), ddPCR, and qPCR.

It is known that mutations in tumor cell-free DNA from liquid biopsy are more sensitive to PCR detection when DNA greater than 1 Kb is removed (See, e.g., WO2009049147 A2). As disclosed herein, this invention involves manipulations of various substrates, such as magnetic beads, to achieve an enriched and purified isolate of cell-free DNA. While a number of methods currently exist to isolate cell-free DNA from bodily fluids, they are not satisfactory in various aspects, such as total yield, removal of high molecular weight species, reproducibility, labor intensiveness, time consumption, level of difficulty, quality of DNA, or adaptability to high-throughput processing.

The invention disclosed herein addresses all of these deficiencies by providing easy-to-use methods and kits for the enrichment of cfDNA for liquid biopsy.

### Methods

Disclosed is a method for the enrichment and purification of cfDNA from a biological specimen. The detection of cfDNA from body fluid (such as urine, plasma, and saliva) has been useful in characterizing aberrations in genomic DNA originating from tumor cells.

In one aspect, the invention herein uses, among others, optimum dosing of EDTA and solid phase reversible immobilization (SPRI) to size select DNA from certain substrates (e.g., silica-based magnetic beads) and further purification to remove PCR inhibitors using other substrates (e.g., carboxylated beads). In some further embodiments, the method can be designed for adaptation to high-throughput robotic automation.

With solid phase reversible immobilization, target nucleic acids are selectively precipitated under specific buffer conditions in the presence of beads or other solid phase materials that are often paramagnetic. The precipitated target nucleic acids immobilize to said beads and remain bound until removed by an elution buffer according to one's needs (see, e.g., DeAngelis et al. (1995) Nucleic Acids Res 23: 4742-4743). In some embodiments, SPRI is used to bind nucleic acids of interest (e.g., cfDNA) to the solid phase and in some embodiments SPRI is used to bind, retain or remove nucleic acids that are not of interest (e.g., HMW DNA) so that the nucleic acids of interest (e.g., LMW DNA or cf-DNA) remain in the non-bound liquid phase (e.g., “reverse SPRI”).

In one example, the present application relates to a method of enriching and purifying cell-free DNA from a biological specimen. The invention in general may include the following:
i. providing a biological sample containing circulating cell-free DNA;
ii. treating the biological sample with EDTA and TRIS buffer to prevent degradation;
iii. concentrating the biological sample if desired to volumes of 1 mL or less;
ii. isolating total DNA from the biological sample;
iii. fractionating the isolated DNA such that DNA 1 Kb or larger in size is preferentially removed;
iv. fractionating the isolated DNA such that DNA equal or less than 1 Kb in size is retained;
iv. eluting the fractionated DNA in a low salt TE buffer.

Sources of nucleic acid samples that can be used include, but are not limited to, human cells such as circulating blood, cultured cells and tumor cells. Other mammalian tissue, blood and cultured cells are suitable sources of template nucleic acids. In addition, viruses, bacteriophage, bacteria, fungi and other micro-organisms can be the source of nucleic acid for analysis. The DNA may be genomic or it may be cloned in plasmids, bacteriophage, bacterial artificial chromosomes (BACs), yeast artificial chromosomes (YACs) or other vectors. The present invention may be used for detection of variation in genomic DNA whether human, animal or other. It finds particular use in the analysis of inherited or acquired diseases or disorders. A particular use is in the detection of inherited diseases and cancer.

In one embodiment, the method comprises collecting a minimally invasive biological fluid from a cancer patient. In another embodiment, the method comprises collecting biological fluid from a pregnant female. In yet another embodiment, the method comprises collecting biological fluid from an individual infected with a virus (such as HBV or HIV) or microorganism. In a further embodiment, the method comprises collecting biological fluid from non-human organisms.

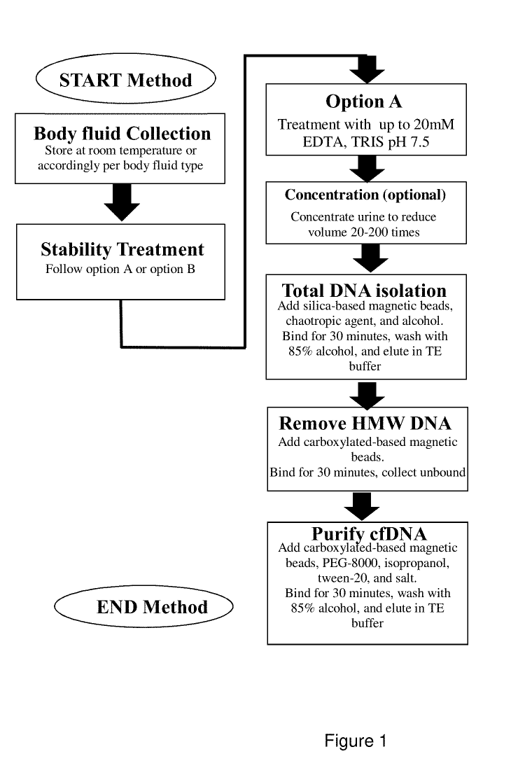

Shown in FIG. 1 is one exemplary method of this invention. The method comprises performing DNA isolation. DNA isolation (or extraction) from a biological sample can be carried out by mixing a chaotropic agent (such as, guanidine thiocyanate, guanidinium chloride, salts, butanol, ethanol, lithium perchlorate, lithium acetate, magnesium chloride, phenol, propanol, sodium dodecyl sulfate, thiourea, and urea) and an alcohol (such as ethanol or isopropanol) in a reaction vesicle with silica-bound magnetic particles (such as PROMEGA's MAGNESIL RED beads, ZINETIX beads, G-BIOSCIENCES Silica Magnetic beads, BIOCLONE's BCMAG™ Silica-modified Magnetic Beads, OCEAN NANOTECH's MONO MAG Silica Beads, etc.).

As mentioned above, EDTA can be used to size select DNA from certain substrates. In some embodiments the total EDTA concentration can be in a range of 5-25 mM (such as 5-20 mM, 7-15 mM), preferable 10 mM, in a DNA isolation mixture. In such a case, the eluted DNA can be subsequently mixed in a secondary DNA isolation reaction vesicle in a solution containing PEG, alcohol, salt and detergent (e.g., with PEG-8000, NaCl, isopropanol, TWEEN-20), and a carboxylated-bound magnetic particle (such as MAGBIO's HIGHPREP PCR beads, BECKMAN COULTER's AMPURE XP, OCEAN NANOTECH's carboxyl MAG beads, etc.).

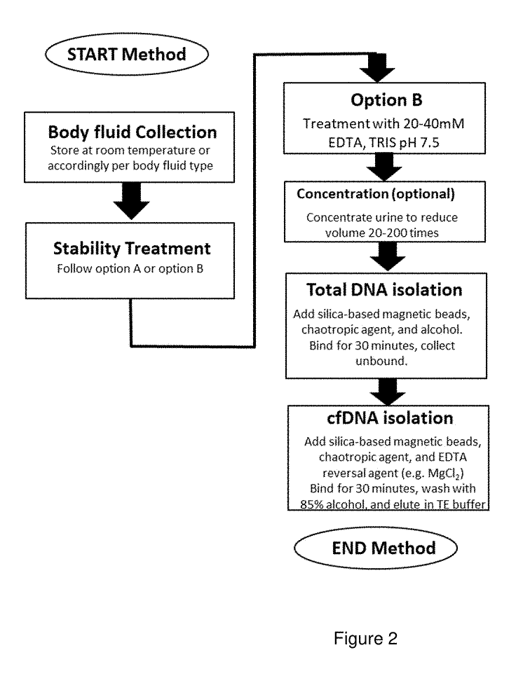

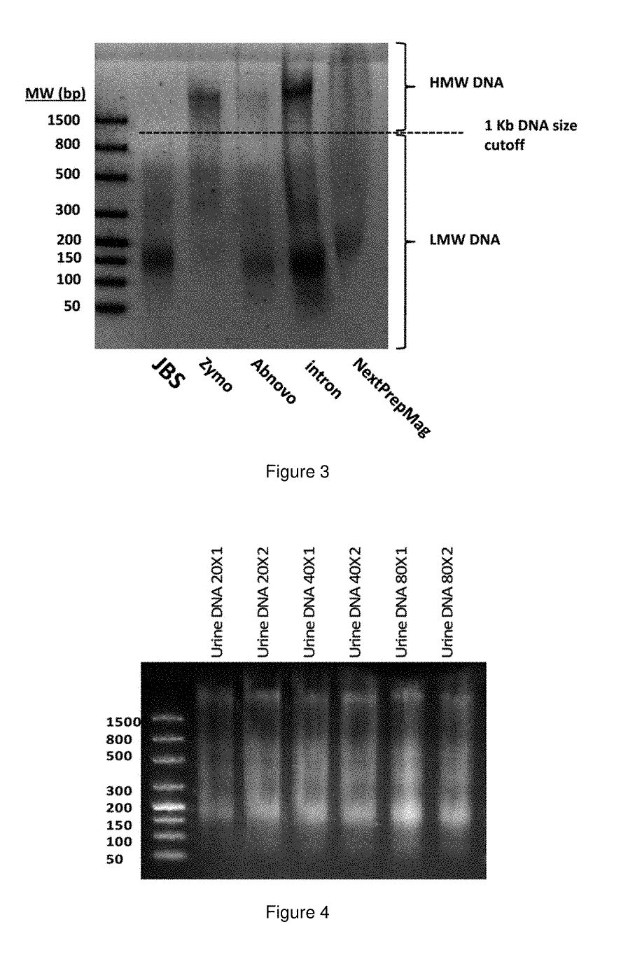

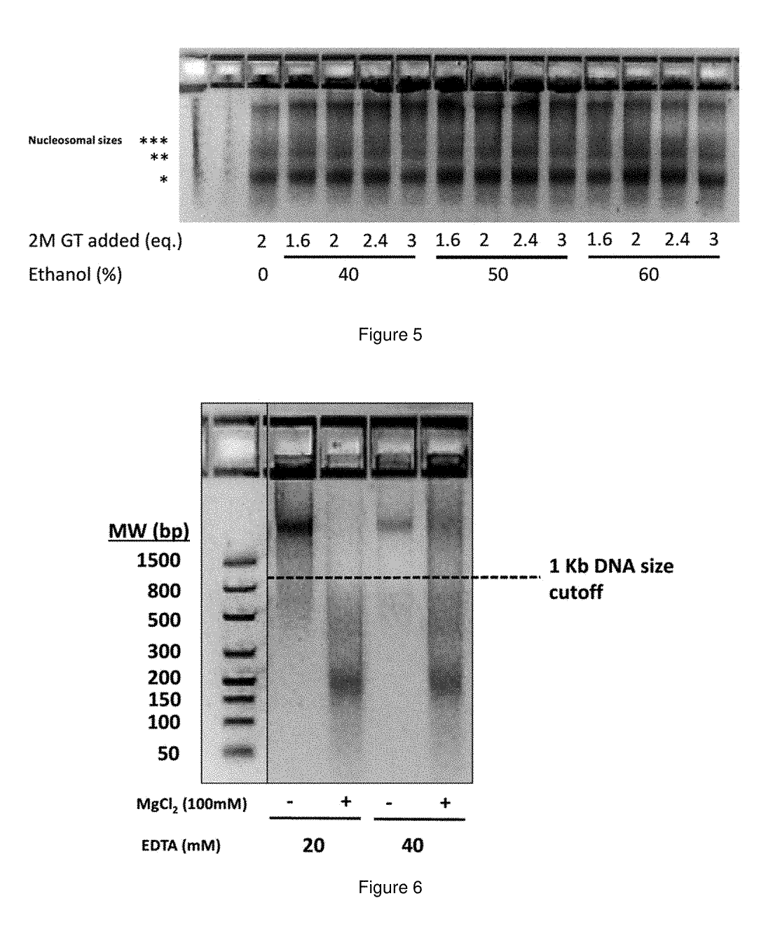

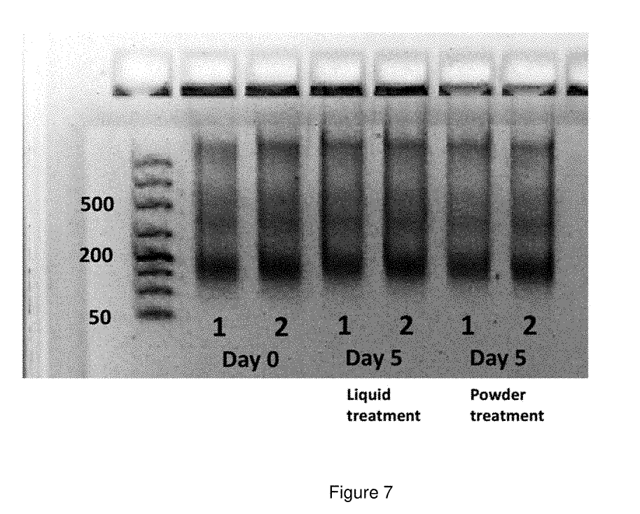

Shown in FIG. 2 is another exemplary method of this invention. In this method, the total EDTA concentration can be adjusted between 20 mM to 40 mM (e.g., 25-35 mM and 30 mM) in a DNA isolation mixture. In such a case, the higher EDTA concentration preferentially allows binding of only high-molecular weight DNA (i.e., DNA larger than 1 kb) to the silica-based magnetic particles, and the unbound solution contains low molecular weight DNA (DNA smaller than 1 kb). Unbound fragments can be recovered by binding to new silica-based magnetic beads and EDTA inhibition reversed by either addition of divalent cations (e.g., magnesium chloride) or decreasing the pH. The bound fragments must be washed with, e.g., 85% ethanol to purify and eluted DNA from the beads in a eluting solution, such as water or a low salt TE buffer.

In the above-described methods, nucleic acids bind non-specifically to substrate (e.g., silica) surfaces in the presence of certain salts and under certain pH conditions, usually under conditions of high ionic strength. For example, DNA adsorption is most efficient in the presence of a buffer solution having a pH at or below the pKa of the surface silanol groups of the silicon surface. In some embodiments, a nucleic acid (e.g., DNA) binds to silica in the presence of a chaotropic agent or chaotrope (e.g., salts, butanol, ethanol, guanidinium chloride, guanidine thiocyanate, lithium perchlorate, lithium acetate, magnesium chloride, phenol, propanol, sodium dodecyl sulfate, thiourea, and urea), which denatures biomolecules by disrupting the shell of hydration around them. In some embodiments, the nucleic acid is washed with high salt and ethanol, and typically eluted with an elution buffer comprising low salt.

The method described herein can be used for various purposes. In one example, the method can be used for early detection of cancer by characterization of altered sequence modification in the enriched cell-free DNA. In another, the method can be used for monitoring efficacy of cancer therapeutics. In another aspect, the method is used for liquid biopsy of circulating DNA for precision medicine. In another example, the method can be used in research settings to characterize tumor genetic and epigenetic modifications present in cell-free DNA that are different than wild-type genomic DNA. In another example, the method can be used to detect DNA sequence or alterations of embryos from the pregnant mothers body fluid. In another example, the method can be used to detect and monitor sequence and/or sequence variations of infectious organisms and particles from body fluid of the infected host.

The methods disclosed in this invention are particularly useful in the areas of (a) early cancer detection from tissue biopsies and bodily fluids such as plasma, serum, or urine; (b) assessment of residual disease after surgery or radiochemotherapy; (c) disease staging and molecular profiling for prognosis or tailoring therapy to individual patients; and (d) monitoring of therapy outcome and cancer remission/relapse.

Cancer can include, but is not limited to, carcinoma, including adenocarcinoma, lymphoma, blastoma, melanoma, sarcoma, leukemia, squamous cell cancer, small-cell lung cancer, non-small cell lung cancer, gastrointestinal cancer, Hodgkin's and non-Hodgkin's lymphoma, pancreatic cancer, glioblastoma, basal cell carcinoma, biliary tract cancer, bladder cancer, brain cancer including glioblastomas and medulloblastomas; breast cancer, cervical cancer, choriocarcinoma; colon cancer, colorectal cancer, endometrial carcinoma, endometrial cancer; esophageal cancer, gastric cancer; various types of head and neck cancers, intraepithelial neoplasms including Bowen's disease and Paget's disease; hematological neoplasms including acute lymphocytic and myelogenous leukemia; Kaposi's sarcoma, hairy cell leukemia; chromic myelogenous leukemia, AIDS-associated leukemias and adult T-cell leukemia lymphoma; kidney cancer such as renal cell carcinoma, T-cell acute lymphoblastic leukemia/lymphoma, lymphomas including Hodgkin's disease and lymphocytic lymphomas; liver cancer such as hepatic carcinoma and hepatoma, Merkel cell carcinoma, melanoma, multiple myeloma; neuroblastomas; oral cancer including squamous cell carcinoma; ovarian cancer including those arising from epithelial cells, sarcomas including leiomyosarcoma, rhabdomyosarcoma, liposarcoma, fibrosarcoma, and osteosarcoma; pancreatic cancer; skin cancer including melanoma, stromal cells, germ cells and mesenchymal cells; prostate cancer, rectal cancer; vulval cancer, renal cancer including adenocarcinoma; testicular cancer including germinal tumors such as seminoma, non-seminoma (teratomas, choriocarcinomas), stromal tumors, and germ cell tumors; thyroid cancer including thyroid adenocarcinoma and medullar carcinoma; esophageal cancer, salivary gland carcinoma, and Wilms' tumors. In some embodiments, the cancer can be lung cancer, such as NSCLC.

### Kits

The disclosure also provides kits and diagnostic systems for conducting amplification, enrichment, and/or for detection of a target sequence. To that end, one or more of the reaction components for the methods disclosed herein can be supplied in the form of a kit for use in the enrichment and detection of a target nucleic acid strand. In such a kit, an appropriate amount of one or more reaction components is provided in one or more containers or held on a substrate (e.g., by electrostatic interactions or covalent bonding).

In one example, the kits include one or more components employed in methods of the invention for isolating, enriching, and purifying cell-free DNA, e.g.:
i. A tube containing a chaotropic agent (e.g., guanidine thiocyanate) mixed with an alcohol (e.g., isopropanol);
ii. A tube containing silica-bound magnetic particles (e.g. PROMEGA'S MAGNESIL RED beads);
iii. A tube containing a washing buffer of 80%-100% ethanol;
iv. A tube containing an elution buffer of low salt TE buffer;
v. A tube containing a solution for low-molecular weight DNA binding or DNA purification, composed of PEG-8000, Sodium Chloride, TRIS pH 7.5, Isopropanol, and TWEEN-20;
vi. A tube containing alcohol (e.g., isopropanol);
vii. A tube containing carboxylated-bound magnetic particles (e.g., MAGBIO's HIGHPREP PCR beads);

In one aspect the kit is designed for the method to be used manually. In another aspect the kit is designed for the method to carried out in high-throughput by robotic automation.

A kit containing reagents for performing amplification or enrichment or sequencing (such as those for NGS or Sanger sequencing) of a target nucleic acid sequence using the methods described herein may include one or more of the followings: one or more adapters, a forward primer, a reverse primer, one or more blockers, a nucleic acid polymerase, extension nucleotides, and detection probes. Examples of additional components of the kits include, but are not limited to, one or more different polymerases, one or more primers that are specific for a control nucleic acid or for a target nucleic acid, one or more probes that are specific for a control nucleic acid or for a target nucleic acid, buffers for polymerization reactions (in IX or concentrated forms), and one or more dyes or fluorescent molecules for detecting polymerization products. The kit may also include one or more of the following components: supports, terminating, modifying or digestion reagents, osmolytes, and an apparatus for detecting a detection probe.

The reaction components used in an amplification and/or detection process may be provided in a variety of forms. For example, the components (e.g., enzymes, nucleotide triphosphates, adaptors, blockers, and/or primers) can be suspended in an aqueous solution or as a freeze-dried or lyophilized powder, pellet, or bead. In the latter case, the components, when reconstituted, form a complete mixture of components for use in an assay.

A kit or system may contain, in an amount sufficient for at least one assay, any combination of the components described herein, and may further include instructions recorded in a tangible form for use of the components. In some applications, one or more reaction components may be provided in pre-measured single use amounts in individual, typically disposable, tubes or equivalent containers. With such an arrangement, the sample to be tested for the presence of a target nucleic acid can be added to the individual tubes and amplification carried out directly. The amount of a component supplied in the kit can be any appropriate amount, and may depend on the target market to which the product is directed. General guidelines for determining appropriate amounts may be found in, for example, Joseph Sambrook and David W. Russell, Molecular Cloning: A Laboratory Manual, 3rd edition, Cold Spring Harbor Laboratory Press, 2001; and Frederick M. Ausubel, Current Protocols in Molecular Biology, John Wiley & Sons, 2003.

The kits of the invention can comprise any number of additional reagents or substances that are useful for practicing a method of the invention. Such substances include, but are not limited to: reagents (including buffers) for lysis of cells, divalent cation chelating agents or other agents that inhibit unwanted nucleases, control DNA for use in ensuring that the enzyme complexes and other components of reactions are functioning properly, DNA fragmenting reagents (including buffers), amplification reaction reagents (including buffers), and wash solutions. The kits of the invention can be provided at any temperature. For example, for storage of kits containing protein components or complexes thereof in a liquid, it is preferred that they are provided and maintained below 0° C., preferably at or below −20° C., or otherwise in a frozen state.

The container(s) in which the components are supplied can be any conventional container that is capable of holding the supplied form, for instance, microfuge tubes, ampoules, bottles, or integral testing devices, such as fluidic devices, cartridges, lateral flow, or other similar devices. The kits can include either labeled or unlabeled nucleic acid probes for use in detection of target nucleic acids. In some embodiments, the kits can further include instructions to use the components in any of the methods described herein, e.g., a method using a crude matrix without nucleic acid extraction and/or purification. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like).

A system of this invention, in addition to containing kit components, may further include instrumentation for conducting an assay, e.g., a luminometer for detecting a signal from a labeled probe.

Instructions, such as written directions or videotaped demonstrations detailing the use of the kits or system of the present invention, are optionally provided with the kit or systems. In a further aspect, the present invention provides for the use of any composition or kit herein, for the practice of any method or assay herein, and/or for the use of any apparatus or kit to practice any assay or method herein. Optionally, the kits or systems of the invention further include software to expedite the generation, analysis and/or storage of data, and to facilitate access to databases. The software includes logical instructions, instructions sets, or suitable computer programs that can be used in the collection, storage and/or analysis of the data. Comparative and relational analysis of the data is possible using the software provided.

All of the above-described methods, reagents, and systems provide a variety of diagnostic tools which permit a liquid (e.g., blood)-based, non-invasive assessment of disease status in a subject. Use of these methods, reagents, and systems in diagnostic tests, which may be coupled with other screening tests, such as a chest X-ray or CT scan, increase diagnostic accuracy and/or direct additional testing. In other aspects, the inventions described herein permit the prognosis of disease, monitoring response to specific therapies, and regular assessment of the risk of recurrence. The inventions described herein also permit the evaluation of changes in diagnostic signatures present in pre-surgery and post therapy samples and identifies a gene expression profile or signature that reflects tumor presence and may be used to assess the probability of recurrence.

A significant advantage of the methods of this invention over existing methods is that they are able to characterize the disease state from a minimally-invasive procedure, e.g., by taking a sample without isolating cancer cells. In contrast current practice for classification of cancer tumors from gene expression profiles depends on a tissue sample, usually a sample from a tumor. In the case of very small tumors, a biopsy is problematic and clearly if no tumor is known or visible, a sample from it is impossible. No purification or isolation of tumor is required, as is the case when tumor samples are analyzed. Urine or blood samples have an additional advantage, which is that the material is easily prepared and stabilized for later analysis.

### Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

As used in this specification and the appended claims, the singular forms “a,” “an,” and “the” include plural referents unless the content clearly dictates otherwise. Thus, for example, reference to “a cell” includes a combination of two or more cells, and the like.

A “nucleic acid” refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA.

As used herein, “cell-free DNA” refers to DNA that is not within a cell. The term “cell-free DNA” includes to any DNA collected from a bodily fluid that originated from a cell that has undergone cell death (e.g., apoptosis) and released its genomic DNA into circulation as fragments. In one embodiment, cell free DNA includes DNA circulating in blood. In another embodiment, cell free DNA includes DNA existing outside of a cell. In yet another embodiment, cell free DNA includes DNA existing outside of a cell as well as DNA present in a blood sample after such blood sample has undergone partial or gentle cell lysing.

It has been demonstrated that urine, as well as other body fluids, contains circulating cell-free (cfDNA). This form of urine DNA, cfDNA, is a fragment of the organisms genomic DNA that may originate from other tissues or organs (for example lung, liver, or stomach). Urine containing cfDNA provides a source of tumor related mutations and alterations that can be used for the detection of cancer-related DNA markers. [3-7].

Circulating cell-free DNA (cfDNA) has been identified in biological fluids [8-10]. For example, in urine, two species are seen: a high-molecular-weight (HMW) DNA, greater than 1 kb, derived mostly from sloughed off cell debris from the urinary tract, and a low-molecular-weight (LMW) DNA, approximately 150 to 250 base pairs (bp), derived primarily from apoptotic cells [6].

Isolation and enrichment of cfDNA from biological fluids that is less than 1 Kb can be used for the detection of cancer related aberrations or for the detection of infectious disease, such as the hepatitis B virus. Purification of cfDNA from larger DNA species is often useful and necessary to provide sensitive and specific detection.

The present invention has the advantage over other DNA isolation methods by removing the larger genomic DNA fragments greater than 1 Kb, which can interfere with detection of DNA or RNA sequence.

The term “nucleotide sequence” and “oligonucleotide” as used herein indicates a polymer of repeating nucleic acids (Adenine, Guanine, Thymine, Cytosine, and Uracil) that is capable of base-pairing with complement sequences through Watson-Crick interactions. This polymer may be produced synthetically or originate from a biological source.

The term “deoxyribonucleic acid” and “DNA” refer to a polymer of repeating deoxyribonucleic acids.

The term “Low molecular weight DNA” refers to any DNA sequence that is less than or equal to 1 Kb in length.

As used herein, “cell-free fetal DNA” (“cffDNA”) refers to DNA that originated from the fetus and not the mother and is not within a cell. In one embodiment, cell free fetal DNA includes fetal DNA circulating in maternal blood. In another embodiment, cell free fetal DNA includes fetal DNA existing outside of a cell, for example a fetal cell. In yet another embodiment, cell free fetal DNA includes fetal DNA existing outside of a cell as well as fetal DNA present in maternal blood sample after such blood sample has undergone partial or gentle cell lysing.

The term “ribonucleic acid” and “RNA” refer to a polymer of repeating ribonucleic acids.

As used herein, the term “target nucleic acid” or “target” refers to a nucleic acid containing a target nucleic acid sequence. A target nucleic acid may be single-stranded or double-stranded, and often is DNA, RNA, a derivative of DNA or RNA, or a combination thereof. A “target nucleic acid sequence,” “target sequence” or “target region” means a specific sequence comprising all or part of the sequence of a single-stranded nucleic acid. A target sequence may be within a nucleic acid template, which may be any form of single-stranded or double-stranded nucleic acid. A template may be a purified or isolated nucleic acid, or may be non-purified or non-isolated.

The term “disease” or “disorder” is used interchangeably herein, and refers to any alteration in state of the body or of some of the organs, interrupting or disturbing the performance of the functions and/or causing symptoms such as discomfort, dysfunction, distress, or even death to the person afflicted or those in contact with a person. A disease or disorder can also relate to a distemper, ailing, ailment, malady, disorder, sickness, illness, complaint, inderdisposion or affectation.

The term “gene” is well known in the art, and herein includes non-coding region such as promoter or other regulatory sequences or proximal non-coding region.

As used herein, the term “subject” refers to any organism having a genome, preferably, a living animal, e.g., a mammal, which has been the object of diagnosis, treatment, observation or experiment. Examples of a subject can be a human, a livestock animal (beef and dairy cattle, sheep, poultry, swine, etc.), or a companion animal (dogs, cats, horses, etc).

A biological sample can comprise of whole tissue, such as a biopsy sample. Other examples of a biological sample comprise biological fluids including, but not limited to, saliva, nasopharyngeal, blood, plasma, serum, gastrointestinal fluid, bile, cerebrospinal fluid, pericardial, vaginal fluid, seminal fluid, prostatic fluid, peritoneal fluid, pleural fluid, urine, synovial fluid, interstitial fluid, intracellular fluid or cytoplasm and lymph, bronchial secretions, mucus, or vitreous or aqueous humor. In other embodiments biological fluid is a research-based sample such as, but not limited to, cell culture and animal studies. In certain embodiments, the preferred biological fluid is urine.

The term “body fluid” or “bodily fluid” refers to any fluid from the body of an animal. Examples of body fluids include, but are not limited to, plasma, serum, blood, lymphatic fluid, cerebrospinal fluid, synovial fluid, urine, saliva, mucous, phlegm and sputum. A body fluid sample may be collected by any suitable method. The body fluid sample may be used immediately or may be stored for later use. Any suitable storage method known in the art may be used to store the body fluid sample: for example, the sample may be frozen at about −20° C. to about −70° C. Suitable body fluids are acellular fluids.

“Acellular” fluids include body fluid samples in which cells are absent or are present in such low amounts that the nucleic acid level determined reflects its level in the liquid portion of the sample, rather than in the cellular portion. Such acellular body fluids are generally produced by processing a cell-containing body fluid by, for example, centrifugation or filtration, to remove the cells. Typically, an acellular body fluid contains no intact cells however, some may contain cell fragments or cellular debris. Examples of acellular fluids include plasma or serum, or body fluids from which cells have been removed.

As used herein, a silica-based substrate (such as a silica-based bead) refers to a material (such as polymer) coated with a silicon dioxide (SiO₂) layer. Since silica is able to bind to nucleic acids, the substrate serve as a simple and efficient tool for DNA purification.

The term “carboxylated” as used herein refers to the modification of a material, such as a microparticle, by the addition of at least one carboxyl group (e.g., COOH or COO—). A carboxylated substrate (e.g., carboxylated beads) refers to a material (such as polymer) coated with surface functional group —COOH/carboxyl molecules. Such a polymer (e.g., a bead made of polystyrene surrounded by a layer of magnetite, which is coated with carboxyl molecules) can reversibly bind DNA in the presence of “crowding agent” such as polyethylene glycol (PEG) and salt (e.g., 20% PEG, 2.5M NaCl). PEG causes the negatively charged DNA to bind with the carboxyl groups on the bead surface. Preferably, the substrates are in the form of columns, membranes, particles, or beads. In particular, the particles or beads can be magnetic to facilitate quick and simple DNA purification. See, e.g., U.S. Pat. No. 8,722,329.

As used herein, the terms “magnetic particles” and “magnetic beads” are used interchangeably and refer to particles or beads that respond to a magnetic field. Typically, magnetic particles comprise materials that have no magnetic field but that form a magnetic dipole when exposed to a magnetic field, e.g., materials capable of being magnetized in the presence of a magnetic field but that are not themselves magnetic in the absence of such a field. The term “magnetic” as used in this context includes materials that are paramagnetic or superparamagnetic materials. The term “magnetic”, as used herein, also encompasses temporarily magnetic materials, such as ferromagnetic or ferrimagnetic materials with low Curie temperatures, provided that such temporarily magnetic materials are paramagnetic in the temperature range at which silica magnetic particles containing such materials are used according to the present methods to isolate biological materials. The term “paramagnetic” as used herein refers to the characteristic of a material wherein said material's magnetism occurs only in the presence of an external, applied magnetic field and does not retain any of the magnetization once the external, applied magnetic field is removed.

As used herein, the term “bead” refers to any type of solid phase particle of any convenient size, of irregular or regular shape, and which is fabricated from any number of known materials such as cellulose, cellulose derivatives, acrylic resins, glass, silica gels, polystyrene, gelatin, polyvinyl pyrrolidone, co-polymers of vinyl and acrylamide, polystyrene cross-linked with divinylbenzene, or the like (as described, e.g., in Merrifield (1964) Biochemistry 3: 1385-1390), polyacrylamides, latex gels, polystyrene, dextran, rubber, silicon, plastics, nitrocellulose, natural sponges, silica gels, controlled pore glass (CPG), metals, cross-linked dextrans (e.g., SEPHADEX), agarose gel (SEPHAROSE), and other solid phase bead supports known to those of skill in the art.

The term “primer” defines an oligonucleotide sequence that is capable of annealing to a complementary target sequence, thereby forming a partially double-stranded region as a starting point from which a polymerase enzyme can continue DNA elongation to create a complementary strand.

The term “diagnosing” means any method, determination, or indication that an abnormal or disease condition or phenotype is present. Diagnosing includes detecting the presence or absence of an abnormal or disease condition, and can be qualitative or quantitative.

The term “genome” and “genomic” refer to any nucleic acid sequences (coding and non-coding) originating from any living or non-living organism or single-cell. These terms also apply to any naturally occurring variations that may arise through mutation or recombination through means of biological or artificial influence. An example is the human genome, which is composed of approximately 3×10⁹ base pairs of DNA packaged into chromosomes, of which there are 22 pairs of autosomes and 1 allosome pair.

Amplification of a selected, or target, nucleic acid sequence may be carried out by a number of suitable methods. See generally Kwoh et al., 1990, Am. Blotechnol. Lab. 8:14-25 [11]. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et; and Sambrook et al., 1989, supra). Preferably, amplifications will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

The terms “express” and “produce” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications.

As used herein, the term “contacting” and its variants, when used in reference to any set of components, includes any process whereby the components to be contacted are mixed into same mixture (for example, are added into the same compartment or solution), and does not necessarily require actual physical contact between the recited components. The recited components can be contacted in any order or any combination (or subcombination), and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C. “Contacting a template with a reaction mixture” includes any or all of the following situations: (i) the template is contacted with a first component of the reaction mixture to create a mixture; then other components of the reaction mixture are added in any order or combination to the mixture; and (ii) the reaction mixture is fully formed prior to mixture with the template.

The term “mixture” as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not spatially distinct. In other words, a mixture is not addressable.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 20” may indicate a range of 18 to 22, and “about 1” may mean from 0.9-1.1. Other meanings of “about” may be apparent from the context, such as rounding off, so, for example “about 1” may also mean from 0.5 to 1.4.

The present invention describes an innovative method enabling the isolation, enrichment, and purification of cfDNA from a body fluid such as urine. Collected urine specimens preferably can be treated with a DNA preservative within a few minutes of collection in order to stabilize the cfDNA and prevent degradation. The preserved urine may then be subjected to concentration, if desired, by such methods as centrifugal concentration, using GE VIVASPIN® 20 Centrifugal concentrators. Filter cut-offs of 5 kDa and higher can be used to concentrate cfDNA, although a cut-off of 10 kDa is optimal. Equivalent amounts of a buffered guanidine thiocyanate and isopropanol mixture must be added to the concentrated or non-concentrated, preserved urine volume, as well as silica-coated magnetic particles such as the PROMEGA MAGNESIL® RED beads to collect total DNA.

Fractionation of cfDNA from total DNA isolation is a critical step to ensure collection of purified cfDNA. This step may be performed in one of two exemplary routes as shown in FIGS. 1 and 2.

In one route (FIG. 2), the EDTA concentration in mix of guanidine, isopropyl alcohol and PROMEGA MAGNESIL® RED beads can be adjusted to about 20-40 mM depending on the desired size selection cut-off. Elution using water or TRIS-EDTA buffer from these silica-coated particles removes the larger DNA (or “HMW”). A second elution with a divalent cation (such as Magnesium chloride) can be used to elute the remaining DNA that contains the enriched cfDNA. Alternatively, decreasing the pH may also be used for the second elution.

In the second route (FIG. 1), the EDTA concentration can be about 20 mM or lower and the total DNA eluted in water or TRIS-EDTA buffer. To this elution a second binding with carboxylated magnetic particles, such as MAGBIO's HIGHPREP™ PCR beads can be added for size selection and further cleanup (or purification) of DNA. To obtain the ideal cutoff of 1 Kb and less 0.285× beads can be used to size select out HMW DNA. The elution from these beads can be added to a new mixture of 20% PEG-8000, 5M NaCl, TRIS pH 7.5, and 0.05% TWEEN-20, following a procedure similar to previously published methods described in Su et al. 2008 Annals of the New York Academy of Sciences, 2008. 1137: p. 82-91.). To this mixture is added isopropyl alcohol and carboxylated magnetic particles to bind the remaining LMW DNA. The beads are washed with 85% ethanol and eluted in water or TRIS-EDTA buffer.

In the presence of high concentrations of EDTA, low molecular weight DNA is unable to bind to silica-based magnetic beads in the presence of guanidine thiocyanate and alcohol.

Chelation of divalent cations (such as Magnesium) by EDTA occurs at a 1:1 molar ratio. Once bound, the effect of EDTA is reversed. Likewise, inhibition of silica-based magnetic bead binding to low molecular weight DNA is reversed.

The invention disclosed herein does not depend on spin columns requiring multiple centrifugation steps, such as those provided in the ZYMO QUICK-DNA™ Urine kit or NEXTPREP-MAG™ Urine cfDNA Isolation Kit. Such steps lead to difficulties in processing large batches of samples or in implementing robotic automation. The invention disclosed herein does not depend on the use of column filtration, such as that provided in the ABNOVO™ Urine DNA Purification Kit. Such steps also can lead to difficulties in processing large batches of samples or in implementing robotic automation.

The invention disclosed herein is suitable for the detection and/or quantification of tumor or infectious DNA modifications by PCR, for diagnosis of disease or monitoring of therapy. The invention has the capability to be adapted to robotic automation, such as those that currently isolate total DNA (for example the PROMEGA MAXWELL® RSC instruments).

### EXAMPLES

### Example 1

This example describes an overall procedure for cfDNA using Option A.

The method begins by obtaining patient or specimen fluid samples that have been treated with a preservative (in liquid or powder form), EDTA in TRIS pH 8.0 (FIG. 1). The fluid can be concentrated to 0.5 mL, if desired. A VIVASPIN® 20 centrifugal concentrator may typically be used in this procedure to concentrate fluid such as urine. To the 0.5 mL (or less) of fluid is added 1 mL of 2 M Guanidine Thiocyanate in 25-75% Isopropanol, and mixed well with the sample. To this mix is added 40 μl of silica-based magnetic beads (e.g., PROMEGA MAGNESIL® RED) and rotated at room temperature for at least 30 minutes. The mixture is placed on a magnet to hold the beads, and the unbound is removed. The beads are washed multiple times with 85% ethanol, dried, and eluted in a low salt TE buffer. To the eluted “total” DNA is added carboxylated-based magnetic beads (e.g., MAGBIO HIGHPREP® PCR) at 0.29× final, and rotated for 30 minutes. The magnetic beads are then placed on a magnet, and the unbound is transferred to a new container with a 0.86 volume equivalent of a reagent composed of 20% PEG-8000, 2.5 mM NaCl, TRIS pH 7.5, and 0.05% TWEEN-20. To this mixture is added 0.15 volume equivalents of carboxylated-based magnetic beads, and finally isopropyl alcohol such that the final volume contains 63.4% isopropyl alcohol. This mixture is rotated for 10 minutes, placed on a magnet to remove the unbound, washed with 85% ethanol, dried for 30 minutes, and eluted in a low salt TE buffer.

### Example 2

This example describes an overall Procedure for cfDNA using Option B.

The method begins by obtaining patient or specimen fluid samples that have been treated with a preservative (in liquid or powder form), EDTA in TRIS pH 8.0 (FIG. 1). The fluid can be concentrated to 0.5 mL, if desired. A VIVASPIN® 20 centrifugal concentrator may be used in this procedure to concentrate fluid such as urine. To the 0.5 mL (or less) of fluid is added 1 mL of 2 M Guanidine Thiocyanate in 25-75% Isopropanol, and mixed well with the sample. To this mix is added 40 μl of a silica-based magnetic bead (e.g., PROMEGA MAGNESIL® RED) and rotated at room temperature for at least 30 minutes. The mixture is placed on a magnet to hold the beads, and the unbound is removed and treated with MgCl2 to reverse the EDTA inhibition of low-molecular weight DNA binding of the beads. Then 40 μl of silica-based magnetic beads (e.g., PROMEGA MAGNESIL® RED) are added and rotated at room temperature for at least 30 minutes. The beads are washed multiple times with 85% ethanol, dried, and eluted in a low salt TE buffer.

### Example 3

In this example, assays were carried out to detect a Y chromosome DNA in a plasma sample from a pregnant female with a male fetus.

Briefly, plasma sample from a pregnant female with a male fetus was spiked with a 107 bp double stranded (ds) HBV PCR product (Genbank accession # NC_003977⋅1; nt 1685-1791). The plasma sample was well mixed and 500 ul aliquots were prepared. DNA was isolated using the QIAAMP Circulating Nucleic Acid Kit (QIAGEN), ZINEXTS Cell free DNA isolation kit (ZINETIXS) and MAGMAX cfDNA isolation kit (MAGMAX, THERMOFISHER) in triplicate according to manufacturers' instructions. One half of the DNA obtained was further cleaned with MAGBIO carboxylated beads.

This further clean-up began by purification of DNA with a 1.6× volume equivalent of a reagent composed of 20% PEG-8000, 2.5 mM NaCl, TRIS pH 7.5, and 0.05% TWEEN-20. To this mixture was added 0.4 volume equivalents of carboxylated-based magnetic beads, and finally isopropyl alcohol such that the final volume contained 60% isopropyl alcohol. This mixture was rotated for 10 minutes, placed on a magnet to remove the unbound, washed with 85% ethanol, dried for 30 minutes, and eluted in a low salt TE buffer.

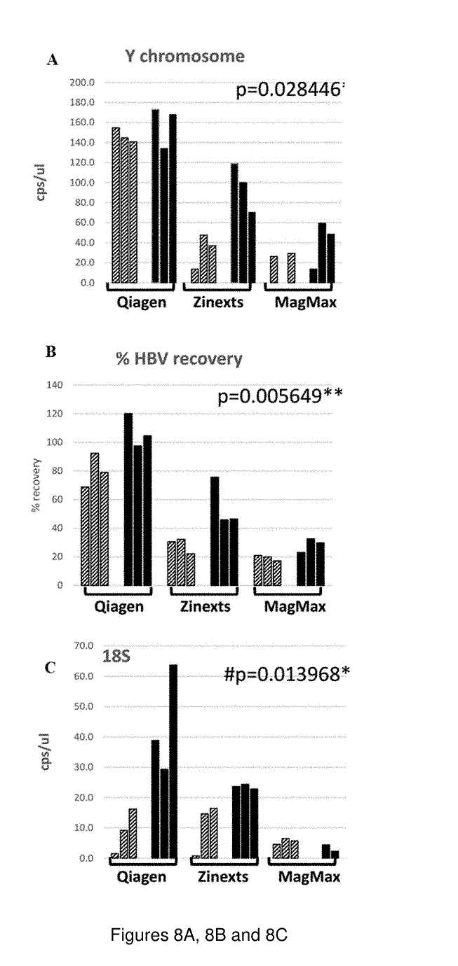

Y chromosome qPCR assay (forward primer, 5′-CATCCAGAGCGTCCCTGGCTT, SEQ ID NO: 1; Genbank accession # NG_016162.2 nt.586-606, reverse primer 5′-GGCCGAAGAAACACTGAGAA SEQ ID NO: 2; Genbank accession # NG_016162.2 nt.626-645), HBV qPCR assay, HBV 1741-1791 (Jain et al. 2018, BMC Gastroenterology (2018) 18:40-48) and 18 s quantitative PCR (Su et. al, 2008, Annals of the New York Academy of Sciences, 2008. 1137: p. 82-91) were performed on each of the plasma DNA before and after cleanup in triplicate by the carboxylated beads. The results are shown in FIGS. 8A, 8B and 8C. It was found that the quantities for all three genes were significantly higher in after cleanup samples (solid) as compared to before cleanup (hatched) by paired student t-test. Since a very low level of 18 s was obtained the MAGMAX kit (below the lower limit of linearity of the qPCR, MAGMAX was excluded from the p value analysis for the quantity of 18 s DNA.

The results indicate that further purification of DNAs isolated from silica-based beads or silica based column with carboxylated beads allowed one to obtain cleaner templates for PCR amplification and enhance PCR efficiency.

### Example 4

In this example, assays were carried out to detect a 107 bp double stranded (ds) HBV PCR product in a human plasma sample.

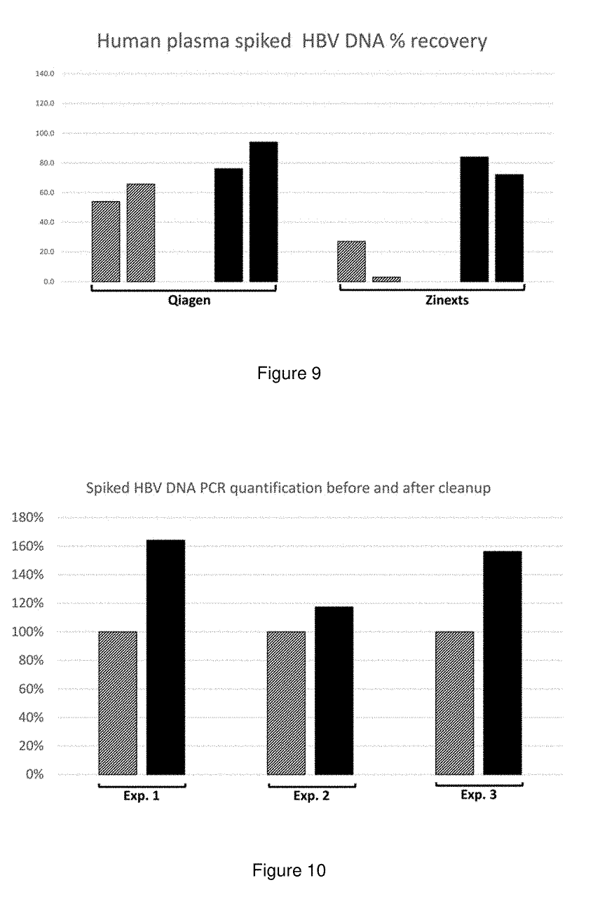

Briefly, one normal human plasma sample was spiked with a 107 bp d) HBV PCR product (Genbank accession # NC_003977⋅1; nt 1685-1791). The plasma sample was well mixed and 500 ul aliquots were prepared. DNA was isolated as per manufacturer's instructions using the QIAAMP Circulating Nucleic Acid Kit (QIAGEN), ZINEXTS Cell free DNA isolation kit in duplicate. One half of the DNA obtained was further cleaned with MAGBIO carboxylated beads according to the procedure described as DNA purification in FIG. 1 and Example 3 above. HBV quantitative PCR assay, HBV 1741-1791 (Jain et al. 2018, BMC Gastroenterology (2018) 18:40-48) was performed on the isolated DNA before and after cleanup. The results are shown in FIG. 9. It was found that the percent recovery of the spiked HBV PCR product as compared to the input DNA was higher in after cleanup samples (solid) as compared to before cleanup (hatched).

The results also indicate that further purification of DNAs isolated from silica-based beads or silica based column with carboxylated beads allowed one to obtain cleaner templates for PCR amplification and enhance PCR efficiency.

### Example 5

In this example, assays were carried out to detect and quantify a 107 bp ds HBV PCR product in human urine samples.

Briefly, three normal human urine samples were spiked with a 107 bp ds) HBV PCR product (Genbank accession # NC_003977⋅1; nt 1685-1791). Total DNA was isolated as described above in Option A (cfDNA isolation, illustrated in FIG. 1). One half of the DNA obtained was further cleaned with MAGBIO carboxylated beads as described above (DNA purification in FIG. 1). HBV quantitative PCR assay, HBV 1741-1791 (Jain et al. 2018, BMC Gastroenterology (2018) 18:40-48) was performed on each of the isolated DNA before and after cleanup.

The results are shown in FIG. 10, where the amount of spiked DNA before cleanup (hatched) is set as 100% for easy comparison. It was found that the quantity of the spiked HBV DNA as measured by HBV 1741-1791 qPCR assay was significant higher after DNA cleanup (solid) as compared to before cleanup (hatched). The results further indicate that additional purification of DNAs isolated from silica-based beads or silica based column with carboxylated beads allowed one to obtain cleaner templates for PCR amplification and enhance PCR efficiency.

### REFERENCES

1. Diaz, L. A. and A. Bardelli, Liquid Biopsies: Genotyping Circulating Tumor DNA. Journal of clinical oncology: official journal of the American Society of Clinical Oncology, 2014. 32(6): p. 579-586.
2. Mouliere, F., et al., Selecting Short DNA Fragments In Plasma Improves Detection Of Circulating Tumour DNA. bioRxiv, 2017.
3. Su, Y.-H., et al., Detection of K-ras mutation in urine of patients with colorectal cancer. Cancer Biomarkers, 2005. 1: p. 177-182.
4. Su, Y.-H., et al., Removal of high molecular weight DNA by carboxylated magnetic beads enhances the detection of mutated K-ras DNA in urine. Annals of the New York Academy of Sciences, 2008. 1137: p. 82-91.
5. Song, B. P., et al., Detection of Hypermethylated Vimentin in Urine of Patients with Colorectal Cancer. Journal of Molecular Diagnostics, 2012. 14(2).
6. Wang, M., et al., Preferential isolation of fragmented DNA enhances the detection of circulating mutated k-ras DNA. Clinical Chemistry, 2004. 50(1): p. 211-213.
7. Lin, S. Y., et al., A locked nucleic acid clamp-mediated PCR assay for detection of a p53 codon 249 hotspot mutation in urine. Journal of Molecular Diagnostics, 2011. 13(5): p. 474-484.
8. Anker, P., et al., Circulating nucleic acids in plasma or serum. Clinica Chimica Acta, 2001. 313: p. 143-146.
9. Chiu, R. W. K., et al., Quantitative Analysis of Circulating Mitochondrial DNA in Plasma. Clinical Chemistry, 2003. 49(5): p. 719.
10. Diehl, F., et al., Circulating mutant DNA to assess tumor dynamics. Nat Med, 2008. 14(9): p. 985-990.
11. KWOH ET AL., AM. BIOTECHNOL. LAB., 1990. 8: p. 14-25.
12. Kwoh, D. Y., et al., Transcription-based amplification system and detection of amplified human immunodeficiency virus type 1 with a bead-based sandwich hybridization format. Proceedings of the National Academy of Sciences of the United States of America, 1989. 86(4): p. 1173-1177.
13. Mamiatis, T., et al., Molecular cloning—A laboratory manual. New York: Cold Spring Harbor Laboratory. 1982, 545 S., 42 $. Acta Biotechnologica, 1985. 5(1): p. 104-104.
14. Lizardi, P. M., et al., Exponential Amplification of Recombinant-RNA Hybridization Probes. Bio/Technology, 1988. 6: p. 1197.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

What is claimed:

1. A method of isolating or enriching circulating cell-free DNA (cfDNA) molecules, comprising:
(a) providing a solution containing DNA molecules;
(b) contacting the solution with a silica-based substrate under a binding condition allowing low molecular weight DNA (LMW DNA) molecules and high molecular weight DNA (HMW DNA) molecules to bind to the silica-based substrate;
(c) eluting the silica-based substrate to obtain an eluate;
(d) contacting the eluate with a first carboxylated-based substrate under a first condition allowing the HMW DNA molecules to bind to the first carboxylated-based substrate; and
(e) obtaining unbound DNA molecules, thereby isolating or enriching the cfDNA molecules.

2. The method of claim 1, wherein said binding condition comprises 5-20 mM EDTA.

3. The method of claim 2, wherein said binding condition further comprises a chaotropic agent or alcohol.

4. The method of claim 1, wherein the obtaining step comprises:
contacting the unbound DNA molecules with a second carboxylated-based substrate under a second condition allowing the cfDNA molecules, not allowing impurity that affecting PCR reaction, to bind to the second carboxylated-based substrate and
eluting the second carboxylated-based substrate to obtain the purified cfDNA molecules.

5. The method of claim 4, wherein the second condition comprises PEG-8000, isopropanol, TWEEN 20, and salt.

6. The method of claim 4, wherein one or more of the substrates are in the form of beads, membrane, or columns.

7. The method of claim 1, wherein the solution is prepared from a biological sample of a subject.

8. The method of claim 7, wherein the biological sample has been concentrated to reduce the volume thereof.

9. The method of claim 7, wherein the biological sample is treated with EDTA and TRIS within a few minutes post collection from the subject.

10. The method of claim 1, wherein eluting the silica-based substrate comprises eluting with water or a low-salt TE buffer.

11. A method of isolating or enriching cfDNA molecules, comprising:
(a) providing a solution containing DNA molecules;
(b) contacting the solution with a first silica-based substrate under a first condition allowing HMW DNA molecules to bind to the silica-based substrate;
(c) obtaining unbound DNA molecules;
(d) contacting the unbound DNA molecules with a second silica-based substrate under a second condition allowing cfDNA molecules to bind to the second silica-based substrate; and
(e) eluting the second silica-based substrate to obtain an eluate, thereby isolating or enriching the cfDNA molecules.

12. The method of claim 11, wherein the first condition comprises 20-40 mM EDTA.

13. The method of claim 12, wherein the first condition further comprises a chaotropic agent or alcohol.

14. The method of claim 11, wherein the second condition is free of EDTA, comprises an EDTA reversal agent, or comprises pH 5-7.

15. The method of claim 14, wherein the second condition further comprises a chaotropic agent.

16. The method of claim 11, wherein one or more of the substrates are in the form of beads, membrane, or columns.

17. The method of claim 11, wherein the solution is prepared from a biological sample of a subject.

18. The method of claim 17, wherein the biological sample has been concentrated to reduce the volume thereof.

19. The method of claim 17, wherein the biological sample is treated with EDTA and TRIS within a few minutes post collection from the subject.

20. A kit for isolating or enriching cfDNA molecules, comprising
(i) a silica-based substrate and
(ii) one or more selected from the group consisting of a carboxylated-based substrate, EDTA, a chaotropic agent, an alcohol, an eluting buffer, PEG, a detergent, and an EDTA reversal agent.
